(19) United States
(12) Patent Application Publication
(10) Pub. No.: US 2009/0087907 A1
(43) Pub. Date: Apr. 2, 2009

(54) Compositions and Methods for Growth of Pluripotent Cells

(76) Inventors: Alice Pebay, Melbourne Tasmania (AU); Anna E. Michalska, Ascot Vale Victoria (AU); Martin F. Pera, Prahran Victoria (AU)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/997,019

(22) PCT Filed: Jul. 31, 2006
(86) PCT No.: PCT/AU2006/001079

§ 371 Date: May 28, 2008

Related U.S. Application Data
(60) Provisional application No. 60/703,752, filed on Jul. 29, 2005.

(51) Int. Cl. C12N 5/10; C07K 14/00; C07H 21/00; C12M 3/00; C12N 1/00; C12N 15/63

(52) U.S. Cl. 435/395; 530/300; 536/23.1; 435/320.1; 435/325; 435/243; 435/289.1

(57) ABSTRACT

A method of propagating embryonic stem (ES) cells in an undifferentiated state, while maintaining both the pluripotency and the cells normal genotype is disclosed. The method comprises using recombinantly produced protein domains to attach human embryonic stem cells to the surface of a bioreactor. The ES cells are supplied with nutrients while they held in place by the recombinantly produced protein domains which may be chosen from Laminin G domain, Fibronectin domain 2, Fibronectin domain 3, Nidogen G2 domain, Nidogen G3 domain, Vitronectin somatomedin B domain, and Vitronectin somatomedin C terminal domain. Useful molecules are characterized by a high binding affinity for hES cells and a molecular weight of about 50 kDa±20%.

### FIELD OF THE INVENTION

This invention relates generally to the field of cell culture media and more particularly to compositions such as cell adhesion molecules useful in growing pluripotent and multipotent animal cells such as embryonic stem cells and adult stem cells.

### BACKGROUND OF THE INVENTION

The propagation of suspension and anchorage dependent cells in hollow fiber bioreactors is variously described in the prior art. In general, known procedures entail the use of bioreactors comprising a plurality of media permeable parallel hollow fibers surrounded by an extracapillary space (ECS). Cell growth medium passed through the hollow fiber lumens permeates the lumen walls to support cell growth in the ECS. See, e.g. U.S. Pat. Nos. 3,821,087; 4,439,322 and Ramsay et al. In Vitro 20:10 (1984).

Animal cells and genetically altered derivatives thereof are often cultivated in bioreactors for the continuous production of vaccines, monoclonal antibodies, and pharmaceutical proteins such as hormones, antigens, tissue type plasminogen activators, and the like. For example, pituitary cells can be cultured in vitro to produce growth hormones; kidney cells can be cultured to produce plasminogen activators; and cultured liver cells have been known to produce hepatitis A antigen.

In these bioreactors, cells are essentially a system of catalysts, and the medium supplies and removes the nutrients and growth inhibiting metabolites. To supply nutrients and remove metabolites, the medium in the bioreactor is changed either intermittently or continuously by fluid flow. However, because of their relatively small size and small density difference when compared to the medium, cells inevitably are withdrawn when the medium is changed, resulting in a relatively low cell concentration within the bioreactor. As a result of this low cell concentration, the concentration of the desired cell product is low in the harvested medium.

An ideal animal cell bioreactor may include three features:

(1) cells would be retained in a viable state at high densities in the bioreactor apparatus for the desired time, with possibly an almost infinite residence time;

(2) high molecular weight compounds, including expensive growth factors and the desired cell products, would have a potentially long but finite residence time within the bioreactor to allow for both efficient nutrient utilization by the growing cells and also the accumulation of cell products to a high concentration; and

(3) low molecular weight compounds, including less expensive nutrients and inhibitory substances, should have a very short residence time within the bioreactor to reduce inhibition of cell growth, cell product formation, and other cellular metabolic activities.

Numerous procedures and devices for in vitro cell culture production of biomolecules have attempted to achieve these goals in the past. In relatively simple systems, the cells have been grown in tissue flasks and roller bottles in the presence of a suitable nutrient media. More complex systems have used capillary hollow fiber membranes as a surface support for the cells in conjunction with a means for supplying nutrient media to the cells.

For example, U.S. Pat. No. 4,537,860 to Tolbert describes a static cell culture maintenance system for maintaining animal cells in a substantially arrested state of proliferation with continuous secretion of cell product. The cells are retained within a reactor vessel chamber in a semi-rigid matrix having interstices for passage of fluid nutrient medium. Fresh nutrient medium is supplied by perfusion into the matrix through relatively low porosity tubes which are suspended in the reactor chamber and which substantially traverse the matrix. High porosity tubes are available to withdraw expended medium and cell product.

A membrane-type cell reactor is also shown in “Construction of a Large Scale Membrane Reactor System with Different Compartments for Cells, Medium and Product”, Develop. Biol. Standard., Vol. 66, pages 221-226 (1987). In this membrane system, cells are immobilized in a wire matrix where different membranes separate the cells from the medium and the cells from the cell product. The membrane lying between the medium and the cells is an ultrafilter with a useful molecular weight cutoff preventing the particular cell product from crossing into the medium compartment. The other membrane is a microfiltration membrane which separates the cells from a cell product chamber. With this configuration it is possible to feed the cells continuously and harvest the collected cell product at a distinct time interval without removing cells.

While these reactor systems attempt to tackle the problems of maintaining a high cell concentration to consequently harvest a high level of cell product, there is much room for improvement particularly with respect to attaching cells to a bioreactor surface. Accordingly, the bioreactor of the present invention provides an in vitro cell culture system which maintains a large number of cells for the required period of time with the possibility of an almost infinite residence time using particular attachment molecules.

### LITERATURE

U.S. Pat. No. 6,703,217; Suzuki et al. (1984) J. Biol. Chem. 259:15307-15314; Kamikubo et al. (2002) J. Biol. Chem. 277:27109-27119; Oldberg and Ruoslahti (1986) J. Biol. Chem. 261:2113-2116; Nomizu et al. (1995) J. Biol. Chem. 270:20583-20590.

### SUMMARY OF THE INVENTION

A method of propagating a pluripotent mammalian cell, e.g., a mammalian embryonic stem (ES) cell, in an undifferentiated state, while maintaining both the pluripotency and the cell's normal genotype is disclosed. The method comprises using recombinantly produced protein domains to attach embryonic stem cells to the surface of a bioreactor. The pluripotent cells are supplied with nutrients while being held in place by the recombinantly produced protein domains which may be chosen from Laminin G domain, Fibronectin domain 2, Fibronectin domain 3, Nidogen G2 domain, Nidogen G3 domain, Vitronectin somatomedin B domain, and Vitronectin somatomedin C terminal domain. Useful molecules are characterized by a high binding affinity for ES cells and a molecular weight of about 50 kDa±20%.

An aspect of the invention is that the protein domain can be produced at a relatively low cost relative to complex multidomain proteins generally isolated from a natural source. The use of separate protein domains makes it easier to present the domains in the correct orientation and provides the ability to readily adjust the concentration of one domain relative to another to achieve optimum attachment. It is also much easier to purify the individual domains.

These and other objects, advantages, and features of the invention will become apparent to those persons skilled in the art upon reading the details of the method and bioreactor system as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 depicts the amino acid sequence of a human laminin al chain G domain (SEQ ID NO.: 1).

FIG. 2 depicts the amino acid sequence of a mouse laminin al chain G domain (SEQ ID NO.:2).

FIG. 3 depicts the amino acid sequence of a rat fibronectin (SEQ ID NO.:3).

FIG. 4 depicts the amino acid sequence of a human fibronectin (SEQ ID NO.:4).

FIG. 5 depicts amino acid sequences of exemplary fibronectin domain 3 cell attachment moieties (SEQ ID NOS.:5-9).

FIG. 6 depicts the amino acid sequence of a human nidogen (SEQ ID NO.: 10).

FIG. 7 depicts the amino acid sequence of a mouse nidogen (SEQ ID NO.:11).

FIG. 8 depicts amino acid sequences of exemplary nidogen G2 and G3 domain cell attachment moieties (SEQ ID NO.: 12-14).

FIG. 9 depicts the amino acid sequence of a human vitronectin (SEQ ID NO.:15).

FIG. 10 depicts the amino acid sequence of an exemplary vitronectin cell attachment moiety (SEQ ID NO.:16).

### DETAILED DESCRIPTION OF PREFERRED EMBODIMENTS

Before the present method of culturing cells and bioreactor are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a,” “an,” and “the” include plural referents unless the context clearly dictates otherwise. Thus, for example, reference to “a molecule” includes a plurality of such molecules and reference to “the substrate surface” includes reference to one or more surfaces and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

“Mammalian Pluripotent Stem Cells” or “pluripotent cells” are pluripotent cells derived from pre-embryonic, embryonic, or fetal tissue of the stated mammalian species at any time after gestation, which have the characteristic of being capable under the right conditions of producing progeny of several different cell types. Generally, pluripotent cells are those capable of producing progeny that are derivatives of all of the three germinal layers: endoderm, mesoderm, and ectoderm, and capable of undergoing proliferation in the absence of feeder cells. Non-limiting examples of primate pluripotent cells are rhesus and marmoset embryonic stem cells, as described by Thompson et al., Proc. Natl. Acad. Sci. U.S.A. 92:7844, 1995, human embryonic stem (hES) cells, as described by Thomson et al., Science 282:1145, 1998; and human embryonic germ (hEG) cells, described in Shamblott et al., Proc. Natl. Acad. Sci. U.S.A. 95:13726, 1998. Other types of non-malignant pluripotent cells are included in the term. Specifically, any cells that are fully pluripotent (that is, they are those capable of producing progeny that are derivatives of all of the three germinal layers) are included, regardless of whether they were derived from embryonic tissue, fetal tissue, or adult tissue.

Pluripotent cell cultures are said to be “substantially undifferentiated” when they display morphology that clearly distinguishes them from differentiated cells of embryo or adult origin. Pluripotent cells typically have high nuclear/cytoplasmic ratios, prominent nucleoli, and compact colony formation with poorly discernable cell junctions, and are easily recognized by those skilled in the art. It is recognized that colonies of undifferentiated cells can be surrounded by neighboring cells that are differentiated. Nevertheless, the substantially undifferentiated colony will persist when cultured under appropriate conditions, and undifferentiated cells constitute a prominent proportion of cells growing upon splitting of the cultured cells. Useful cell populations described in this disclosure contain any proportion of substantially undifferentiated pluripotent having these criteria. Substantially undifferentiated cell cultures may contain at least about 20%, 40%, 60%, or even 80% undifferentiated pluripotent cells (in percentage of total cells in the population).

The term “embryonic stem cell,” as used herein, refers to a cell derived from a group of cells referred to as the inner cell mass (ICM), which ICM is part of the early embryo referred to as the blastocyst. Embryonic stem (ES) cells are pluripotent, e.g., ES cell have the ability to give rise to types of cells that develop from the three germ layers (mesoderm, endoderm, and ectoderm). ES cells include ES cells from any mammal, including primates, e.g., humans, monkeys, apes; ungulates, e.g., horse, pig, cow, sheep, goat; rodents, e.g., mouse, rat; felines; canines; and the like.

A “nutrient medium” or a “culture medium” is a medium for culturing cells containing nutrients that promote proliferation. The nutrient medium may contain any of the following in an appropriate combination: isotonic saline, buffer, amino acids, antibiotics, serum or serum replacement, and exogenously added factors.

The term “polypeptide” refers to a polymer of amino acids and does not refer to a specific length of the product; thus, peptides, oligopeptides, and proteins are included within the definition of polypeptide. This term also does not refer to or exclude post-translational modifications of the polypeptide, for example, glycosylations, acetylations, phosphorylations and the like. Included within the term “polypeptide” are, for example, polypeptides containing one or more analogs of an amino acid (including, for example, unnatural amino acids, non-coded amino acids, etc.), polypeptides with substituted linkages, as well as other modifications known in the art, both naturally occurring and non-naturally occurring. The term “polypeptide” includes fusion proteins, including, but not limited to, fusion proteins with a heterologous amino acid sequence, fusions with heterologous and homologous leader sequences, with or without N-terminal methionine residues; immunologically tagged proteins; and the like.

The terms “polynucleotide” and “nucleic acid” are used interchangeably herein to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes single-, double-stranded and triple helical molecules. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as oligomers or oligos and may be isolated from genes, or chemically synthesized by methods known in the art. The term “polynucleotide” includes double-stranded DNA found, inter alia, in linear DNA molecules (e.g., restriction fragments), viruses, plasmids, and chromosomes.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST. See, e.g., Altschul et al. (1990), J. Mol. Biol. 215:403-10. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

“Recombinant,” as used herein in the context of a nucleic acid, refers to a nucleic acid that is the product of various combinations of cloning, restriction, and/or ligation steps resulting in a construct having a structural coding or non-coding sequence distinguishable from homologous sequences found in natural systems. Generally, DNA sequences encoding the structural coding sequence can be assembled from cDNA fragments and short oligonucleotide linkers, or from a series of oligonucleotides, to provide a synthetic gene which is capable of being expressed in a recombinant transcriptional unit. Such sequences can be provided in the form of an open reading frame uninterrupted by internal nontranslated sequences, or introns, which are typically present in eukaryotic genes. Genomic DNA comprising the relevant sequences could also be used. Sequences of non-translated DNA may be present 5′ or 3′ from the open reading frame, where such sequences do not interfere with manipulation or expression of the coding regions.

The term “conservative amino acid substitution” refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acids substitution groups are: valine-leucine-isoleucine, phenylalanine-tyrosine, lysine-arginine, alanine-valine, and asparagine-glutamine.

“Synthetic nucleic acids” can be assembled from oligonucleotide building blocks that are chemically synthesized using procedures known to those skilled in the art. These building blocks are ligated and annealed to form gene segments which are then enzymatically assembled to construct the entire gene. “Chemically synthesized,” as related to a sequence of DNA, means that the component nucleotides were assembled in vitro. Manual chemical synthesis of DNA may be accomplished using well-established procedures, or automated chemical synthesis can be performed using one of a number of commercially available machines. The nucleotide sequence of the nucleic acids can be modified for optimal expression based on optimization of nucleotide sequence to reflect the codon bias of the host cell. The skilled artisan appreciates the likelihood of successful expression if codon usage is biased towards those codons favored by the host. Determination of preferred codons can be based on a survey of genes derived from the host cell where sequence information is available.

The term “host cell” includes an individual cell or cell culture, which can be or has been a recipient of any recombinant vector(s) or synthetic or exogenous polynucleotide. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a synthetic or exogenous polynucleotide. A host cell which comprises a recombinant vector of the invention is a “recombinant host cell.” In some embodiments, a host cell is a prokaryotic cell. In other embodiments, a host cell is a eukaryotic cell.

The terms “DNA regulatory sequences,” and “regulatory elements,” used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

The term “transformation” is used interchangeably herein with “genetic modification” and refers to a permanent or transient genetic change induced in a cell following introduction of new nucleic acid (i.e., DNA exogenous to the cell). Genetic change (“modification”) can be accomplished either by incorporation of the new DNA into the genome of the host cell, or by transient or stable maintenance of the new DNA as an episomal element. Where the cell is a mammalian cell, a permanent genetic change is generally achieved by introduction of the DNA into the genome of the cell.

The term “operably linked,” as used herein, refers to a juxtaposition wherein the components so described are in a relationship permitting them to function in their intended manner. For instance, a promoter is operably linked to a coding sequence if the promoter effects transcription or expression of the coding sequence.

The term “construct,” as used herein, refers to a recombinant nucleic acid, generally recombinant DNA, that has been generated for the purpose of the expression of a specific nucleotide sequence(s), or is to be used in the construction of other recombinant nucleotide sequences.

As used herein, the term “isolated,” in the context of a nucleic acid, is meant to describe a nucleic acid that is in an environment different from that in which the nucleic acid naturally occurs, or that is in an environment different from that which the nucleic acid was found. As used herein, an “isolated” nucleic acid is one that is substantially free of the nucleic acids or other macromolecules with which it is associated in nature. By substantially free is meant at least 50%, preferably at least 70%, more preferably at least 80%, and even more preferably at least 90% free of the materials with which it is associated in nature. As used herein, an “isolated” nucleic acid also refers to recombinant nucleic acids, which, by virtue of origin or manipulation: (1) are not associated with all or a portion of a nucleic acid with which it is associated in nature, (2) are linked to a nucleic acid other than that to which it is linked in nature, or (3) does not occur in nature.

### The Invention in General

The present invention provides a method of propagating pluripotent cells (e.g., embryonic stem (ES) cells) or multipotent cells (eg adult stem cells or haematopoietic stem cells) in an undifferentiated state, while maintaining both the potency and the cells' normal genotype. The method generally involves attaching a pluripotent cell, such as an ES cell, or a multipotent cell to a surface of a bioreactor using synthetic attachment polypeptides. The present invention further provides synthetic attachment polypeptides; polynucleotides encoding the synthetic attachment polypeptides; and compositions comprising the synthetic attachment polypeptides. The present invention further provides an insoluble support comprising a subject synthetic attachment polypeptide attached thereto. The present invention further provides a bioreactor comprising synthetic attachment polypeptides.

### Synthetic Attachment Polypeptides

The present invention provides synthetic attachment polypeptides, which attachment polypeptides provide for attachment of a pluripotent cell, such as an ES cell, to a solid substrate. A subject synthetic attachment polypeptide comprises a cell surface attachment moiety; and a solid substrate attachment moiety. In some embodiments, a subject synthetic attachment polypeptide comprises the formula:

NH₂—(X₁)ₙ-A-(X₂)ₘ—B—(X₃)ₚ FORMULA I

where A is a moiety that provides for attachment to the surface of a pluripotent cell, such as an ES cell; B is a moiety that provides for attachment to a solid substrate; and X₁, X₂, and X₃ are independently any amino acid, and where n, m, and p are independently 0 or an integer from 1 to about 50, e.g., X₁, X₂, and X₃ are independently from about 1 amino acid to about 5 amino acids, from about 5 amino acids to about 10 amino acids, from about 10 amino acids to about 15 amino acids, from about 15 amino acids to about 20 amino acids, from about 20 amino acids to about 25 amino acids, from about 25 amino acids to about 30 amino acids, from about 30 amino acids to about 35 amino acids, from about 35 amino acids to about 40 amino acids, from about 40 amino acids to about 45 amino acids, or from about 45 amino acids to about 50 amino acids in length. In other embodiments, a subject synthetic attachment polypeptide comprises the formula:

(X₁)ₙ—B—(X₂)ₘ-A-(X₃)ₚ—NH₂ FORMULA II

where A is a moiety that provides for attachment to the surface of a pluripotent cell, such as an ES cell; B is a moiety that provides for attachment to a solid substrate; and X₁, X₂, and X₃ are independently any amino acid, and where n, m, and p are independently 0 or an integer from 1 to about 50, e.g., X₁, X₂, and X₃ are independently from about 1 amino acid to about 5 amino acids, from about 5 amino acids to about 10 amino acids, from about 10 amino acids to about 15 amino acids, from about 15 amino acids to about 20 amino acids, from about 20 amino acids to about 25 amino acids, from about 25 amino acids to about 30 amino acids, from about 30 amino acids to about 35 amino acids, from about 35 amino acids to about 40 amino acids, from about 40 amino acids to about 45 amino acids, or from about 45 amino acids to about 50 amino acids in length.

A subject synthetic attachment polypeptide provides for reversible attachment of a pluripotent cell, such as an ES cell, or a multipotent cell to a solid substrate (“support surface”). A pluripotent cell, such as an ES cell, or a multipotent cell binds to the cell attachment moiety of a subject synthetic attachment polypeptide with a binding affinity of at least about 10⁻⁵ M, at least about 10⁻⁶ M, at least about 5×10⁻⁶ M, at least about 10⁻⁷ M, at least about 5×10⁻⁷ M, at least about 10⁻⁸ M, at least about 5×10⁻⁸ M, or at least about 10⁻⁹ M, or greater.

Attachment of an embryonic or adult stem cell to a solid substrate via the cell attachment moiety of a subject synthetic attachment polypeptide is reversible by alteration of media conditions, e.g., change in pH; addition of soluble peptides that compete with the pluripotent cell, e.g., an ES cell, or a multipotent cell for binding to the cell attachment moiety; and the like.

The solid substrate binding moiety of a subject synthetic attachment polypeptide binds to a solid substrate with binding affinity of at least about 10⁻⁵ M, at least about 10⁻⁶ M, at least about 5×10⁻⁶ M, at least about 10⁻⁷ M, at least about 5×10⁻⁷ M, at least about 10⁻⁸ M, at least about 5×10⁻⁸ M, or at least about 10⁻⁹ M, or greater. In some embodiments, the binding of the solid substrate attachment moiety to a solid substrate is substantially irreversible, e.g., under normal conditions (e.g., under normal cell culture conditions), the binding is irreversible.

In some embodiments, (X₂)ₘ is a linker moiety. Thus, in some embodiments, a cell attachment moiety is coupled to a solid substrate attachment moiety via a linker peptide, which may be cleavable. The linker peptide may have any of a variety of amino acid sequences. Proteins can be joined by a spacer peptide, generally of a flexible nature, although other chemical linkages are not excluded. Suitable linker sequences will generally be peptides of between about 5 and about 50 amino acids in length, or between about 6 and about 25 amino acids in length. These linkers are generally produced by using synthetic, linker-encoding oligonucleotides to couple the proteins. Peptide linkers with a degree of flexibility will generally be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use according to the present invention. Suitable linker peptides frequently include amino acid sequences rich in alanine and proline residues, which are known to impart flexibility to a protein structure. Exemplary linkers for use in a subject synthetic attachment polypeptide have a combination of glycine, alanine, proline and methionine residues, such as AAAGGM (SEQ ID NO:17); AAAGGMPPAAAGGM (SEQ ID NO:18); AAAGGM (SEQ ID NO:19); and PPAAAGGM2 (SEQ ID NO:20). Other exemplary linker peptides include IEGR (SEQ ID NO:21; which can be cleaved by factor Xa); and GGKGGK (SEQ ID NO:22). However, any flexible linker generally between about 5 and about 50 amino acids in length may be used. Linkers may have virtually any sequence that results in a generally flexible peptide, including alanine-proline rich sequences of the type exemplified above.

In some embodiments, a cell attachment moiety is coupled to a solid substrate attachment moiety via a linker peptide that is cleavable by an enzyme. In some embodiments, the enzyme is conditionally activated under a particular physiological condition.

A subject synthetic attachment polypeptide may be synthesized chemically or enzymatically, may be produced recombinantly, may be isolated from a natural source, or a combination of the foregoing. Peptides may be isolated from natural sources using standard methods of protein purification known in the art, including, but not limited to, high-performance liquid chromatography, exclusion chromatography, gel electrophoresis, affinity chromatography, or other purification technique. One may employ solid phase peptide synthesis techniques, where such techniques are known to those of skill in the art. See Jones, The Chemical Synthesis of Peptides (Clarendon Press, Oxford)(1994). Generally, in such methods a peptide is produced through the sequential additional of activated monomeric units to a solid phase bound growing peptide chain. Well-established recombinant DNA techniques can be employed for production of peptides.

### Cell Attachment Moiety

Suitable cell attachment moieties (moieties for attachment of a pluripotent cell, such as an ES cell) are typically from about 10 amino acids in length to about 500 amino acids in length, e.g., from about 10 amino acids to about 25 amino acids, from about 25 amino acids to about 50 amino acids, from about 50 amino acids to about 75 amino acids, from about 75 amino acids to about 100 amino acids, from about 100 amino acids to about 150 amino acids, from about 150 amino acids to about 200 amino acids, from about 200 amino acids to about 250 amino acids, from about 250 amino acids to about 300 amino acids, from about 300 amino acids to about 350 amino acids, from about 350 amino acids to about 400 amino acids, from about 400 amino acids to about 450 amino acids, or from about 450 amino acids to about 500 amino acids in length.

In some embodiments, the cell attachment moiety comprises an amino acid sequence found in a naturally occurring extracellular matrix (ECM) or other cell adhesion molecule. In some embodiments, the cell attachment moiety comprises an amino acid sequence that is a variation of an amino acid sequence found in a naturally occurring extracellular matrix (ECM) or other cell adhesion molecule. In some embodiments, the cell attachment moiety comprises an amino acid sequence found in a polypeptide selected from laminin, fibronectin, nidogen, and vitronectin.

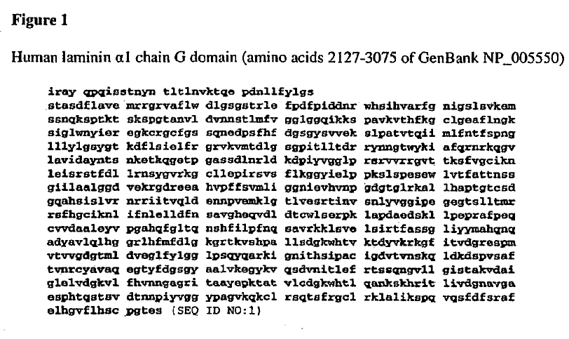

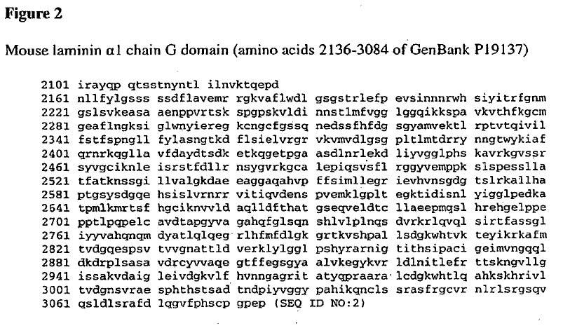

In some embodiments, the cell attachment moiety comprises an amino acid sequence derived from the G domain of laminin, including any isoform of laminin. In particular, in some embodiments, the cell attachment moiety comprises an amino acid sequence derived from the G domain of the laminin α1 chain. In some embodiments, the cell attachment moiety comprises an amino acid sequence of from about 10 to about 500 amino acids, e.g., from about 10 to about 25, from about 25 to about 50, from about 50 to about 75, from about 75 to about 100, from about 100 to about 150, from about 150 to about 200, from about 200 to about 250, from about 250 to about 300, from about 300 to about 350, from about 350 to about 400, from about 400 to about 450, or from about 450 to about 500 contiguous amino acids of the amino acid sequence set forth in SEQ ID NO:1 (depicted in FIG. 1; corresponding to amino acids 2127-3075 of the sequence provide in GenBank Accession No. NP_{—}005550). In some embodiments, the cell attachment moiety comprises an amino acid sequence of from about 10 to about 500 amino acids, e.g., from about 10 to about 25, from about 25 to about 50, from about 50 to about 75, from about 75 to about 100, from about 100 to about 150, from about 150 to about 200, from about 200 to about 250, from about 250 to about 300, from about 300 to about 350, from about 350 to about 400, from about 400 to about 450, or from about 450 to about 500 contiguous amino acids of the amino acid sequence set forth in SEQ ID NO:2 (FIG. 2; corresponding to amino acids 2136-3084 of GenBank P19137).

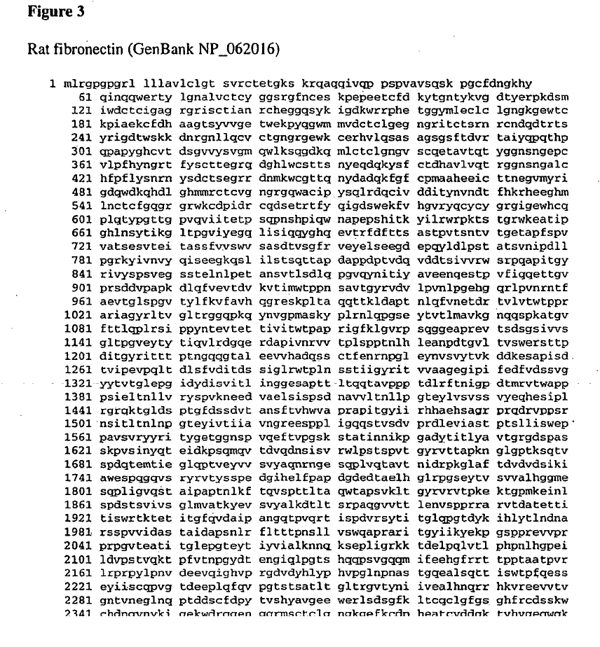

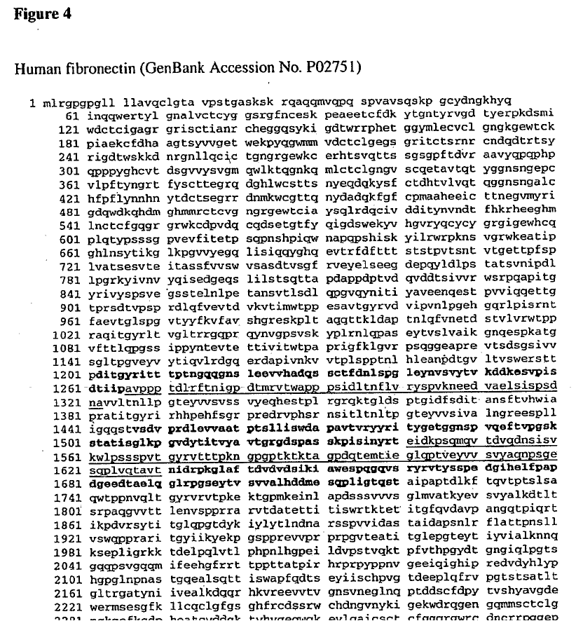

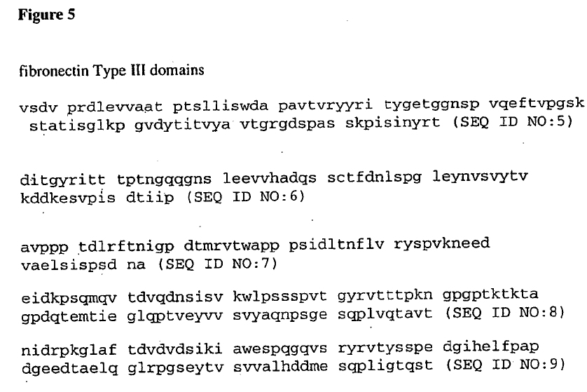

In some embodiments, the cell attachment moiety comprises an amino acid sequence derived from fibronectin. In some embodiments, the cell attachment moiety comprises an amino acid sequence corresponding to a cell-attachment domain found in SEQ ID NO:3 (FIG. 3; Rat fibronectin; GenBank NP_{—}062016). In some embodiments, the cell attachment moiety comprises an amino acid sequence corresponding to a cell-attachment domain found in SEQ ID NO:4 (FIG. 4; human fibronectin; GenBank Accession No. P02751). In some embodiments, the cell attachment moiety comprises an amino acid sequence corresponding to a fibronectin domain 2 (a Type II fibronectin domain). In some embodiments, the cell attachment moiety comprises an amino acid sequence corresponding to a fibronectin domain 3 (a Type III fibronectin domain). In some embodiments, the cell attachment moiety comprises the amino acid sequence set forth in any one of SEQ ID NOS:5-9 (FIG. 5; see, e.g., FIGS. 3 and 4 of Oldberg and Ruoslahti (1986) J. Biol. Chem. 261:2113-2116).

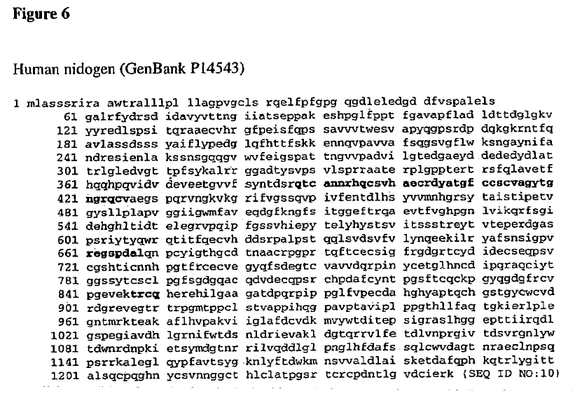

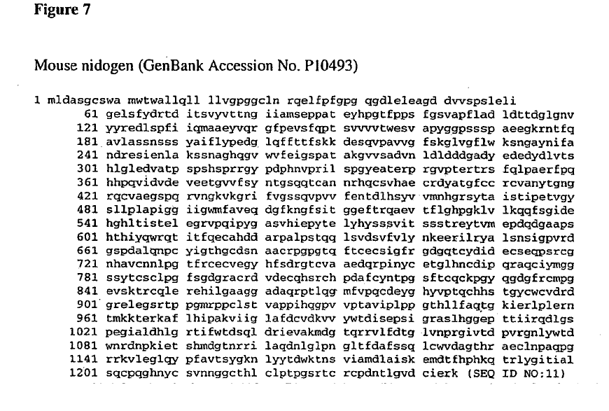

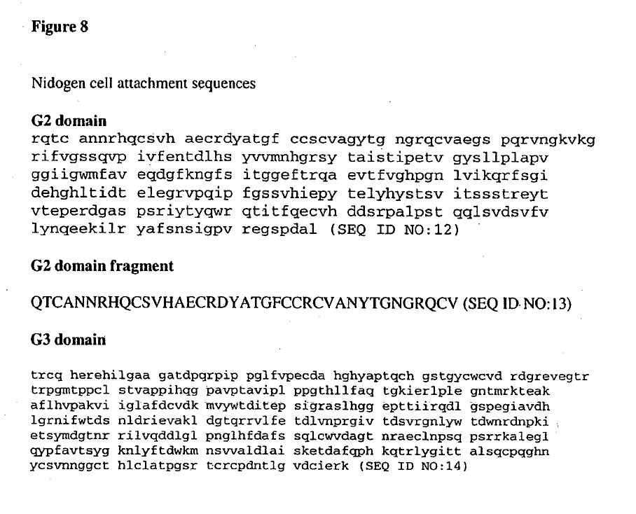

In some embodiments, the cell attachment moiety comprises an amino acid sequence derived from nidogen (also referred to as entactin) G2 and G3 domain. In some embodiments, the cell attachment moiety comprises an amino acid sequence corresponding to a cell-attachment domain found in SEQ ID NO:10 (FIG. 6; human nidogen; GenBank Accession No.P14543). In some embodiments, the cell attachment moiety comprises an amino acid sequence corresponding to a cell-attachment domain found in SEQ ID NO:11 (FIG. 7; mouse nidogen; GenBank Accession No. P10493). In some embodiments, the cell attachment moiety comprises an amino acid sequence corresponding to a G2 domain of nidogen. In some embodiments, the cell attachment moiety comprises an amino acid sequence as set forth in SEQ ID NO:12 (FIG. 8). In some embodiments, the cell attachment moiety comprises an amino acid sequence as set forth in SEQ ID NO:13 (FIG. 8). In some embodiments, the cell attachment moiety comprises an amino acid sequence corresponding to a G3 domain of nidogen. In some embodiments, the cell attachment moiety comprises an amino acid sequence as set forth in SEQ ID NO:14 (FIG. 8).

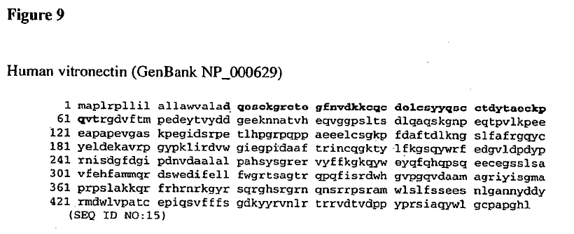

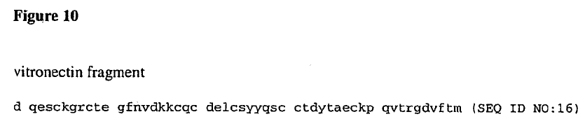

In some embodiments, the cell attachment moiety comprises an amino acid sequence derived from vitronectin. In some embodiments, the cell attachment moiety comprises an amino acid sequence corresponding to a cell-attachment domain found in SEQ ID NO:15 (FIG. 9; human vitronectin; GenBank Accession No. NP_{—}000629). In some embodiments, the ES cell attachment moiety comprises an amino acid sequence derived from vitronectin somatomedin B domain. In some embodiments, the ES cell attachment moiety comprises the amino acid sequence DQESC KGRCT EGFNV DKKCQ CDELC SYYQS CCTDY TAECK PQVT (FIG. 10; SEQ ID NO:16; amino acids 20-63 of the amino acid sequence provided in GenBank NP_{—}000629). In some embodiments, the ES cell attachment moiety comprises an amino acid sequence derived from a vitronectin carboxyl-terminal domain.

In some embodiments, the cell attachment moiety of a subject synthetic attachment polypeptide comprises a functional fragment of a cell attachment moiety selected from a laminin G domain, a fibronectin domain 2, a fibronectin domain 3, a nidogen G2 domain, a nidogen G3 domain, a vitronectin somatomedin B domain, a vitronectin carboxyl-terminal domain and combinations thereof. A “functional fragment” is a fragment that retains the ability to bind a pluripotent cell (e.g., an ES cell) or a multipotent cell with high affinity. Those skilled in the art can readily determine whether a fragment retains the ability to bind a pluripotent cell (e.g., an ES cell) or a multipotent cell with high affinity.

In some embodiments, the cell attachment moiety of a subject synthetic attachment polypeptide comprises an amino acid sequence that comprises from about 1 to about 15 amino acid substitutions, compared to the amino acid sequence of a naturally-occurring cell attachment moiety selected from a laminin G domain, a fibronectin domain 2, a fibronectin domain 3, a nidogen G2 domain, a nidogen G3 domain, a vitronectin somatomedin B domain, and a vitronectin carboxyl-terminal domain.

In some embodiments, the cell attachment moiety of a subject synthetic attachment polypeptide comprises an amino acid sequence that comprises from about 1 to about 15 conservative amino acid substitutions, compared to the amino acid sequence of a naturally-occurring cell attachment moiety selected from a laminin G domain, a fibronectin domain 2, a fibronectin domain 3, a nidogen G2 domain, a nidogen G3 domain, a vitronectin somatomedin B domain, and a vitronectin carboxyl-terminal domain.

In some embodiments, a subject synthetic attachment polypeptide comprises two or more different cell attachment moieties. For example, in some embodiments a subject synthetic attachment polypeptide comprises a laminin G domain (or a functional fragment thereof) and a fibronectin domain 2. In other embodiments, a subject synthetic attachment polypeptide comprises a laminin G domain (or a functional fragment thereof) and a fibronectin domain 3. In other embodiments, a subject synthetic attachment polypeptide comprises a nidogen G2 domain and a laminin G domain (or a functional fragment thereof). In other embodiments, a subject synthetic attachment polypeptide comprises a nidogen G3 domain and a laminin G domain (or a functional fragment thereof). In a preferred embodiment the synthetic attachment polypeptide comprises a combination of domains from laminin, vitronectin and fibronectin.

### Support Surface Attachment Moiety

As discussed above, a subject synthetic attachment polypeptide comprises a cell attachment moiety and a support surface (also referred to as “solid substrate”) attachment moiety. The solid substrate attachment moiety is in many embodiments an organic molecule that provides for covalent linkage to a support surface. In other embodiments, the solid substrate attachment moiety provides for non-covalent attachment of a subject synthetic attachment polypeptide to a support surface. The support surface is in some embodiments derivatized for coupling to the support surface attachment moiety.

Support surfaces include, but are not limited to, dextran, polyacrylamide, nylon, polystyrene, calcium alginate, glass, silica, silicon, collagen, hydroxyapatite, hydrogels, PTFE, polypropylene, nylon, polyacrylamide, and agar gel structures. Support surfaces are in any of a variety of forms, including, but not limited to, beads (e.g., microcarrier beads); plates (e.g., culture dishes, microtiter plates, and the like); membranes (e.g., synthetic membranes); fibers (e.g., hollow fibers); and the like. Suitable support surfaces include particles, e.g., a bead, a microsphere, a nanoparticle, or a colloidal particle. Particle and bead sizes may also be chosen and may have a variety of sizes including wherein the bead is about 5 nanometers to about 500 microns in diameter.

In some embodiments, the support surface is contained within a bioreactor. For example, in some embodiments, the support surface comprises microcarrier beads suspended in a rigid or semi-rigid matrix which is placed within a culture bioreactor. The matrix possesses interstitial passages for the transport of liquid nutrient media into the bioreactor, similarly disposed passages for the outflow of liquid media and product chemicals, and similar interstitial passages through which input and output gases may flow. Bioreactors of this type include the vat type, the packed-column type, and the porous ceramic-matrix type bioreactor. Such bioreactors are taught, for example, in U.S. Pat. Nos. 4,203,801; 4,220,725; 4,279,753; 4,391,912; 4,442,206; 4,537,860; 4,603,109; 4,693,983; 4,833,083; 4,898,718; and 4,931,401.

In other embodiments, a support surface is a membrane of a bioreactor, e.g., a bioreactor in which cells are confined between two synthetic membranes. Typically, one membrane is microporous and hydrophilic and in contact with the aqueous nutrient media, while the opposing membrane is ultraporous and hydrophobic and in contact with a flow of air or an oxygen-enriched gas. Such a configuration provides the cells with an environment in which nutrient liquid input and waste liquid output can occur through channels separate from the cell-containing space and similarly provide gaseous input and output through similarly disposed channels, again separate from the cell-containing space. In some embodiments, the support surface comprises stacks of a plurality of flat membranes forming a multiplicity of cell compartments, e.g., a series of synthetic membrane bags, one within the other, or spirally-wound membrane configurations. Such synthetic membrane support surfaces are taught, for example, in U.S. Pat. Nos. 3,580,840; 3,843,454; 3,941,662; 3,948,732; 4,225,671; 4,661,455; 4,748,124; 4,764,471; 4,839,292; 4,895,806; and 4,937,196.

Another suitable support surface are capillary hollow fibers (usually configured in elongated bundles of many fibers) having micropores in the fiber walls, which are used in bioreactor devices. Typically, cells are cultured in a closed chamber into which the fiber bundles are placed. Nutrient aqueous solutions flow freely through the capillary lumen and the hydrostatic pressure of this flow results in an outward radial perfusion of the nutrient liquid into the extracapillary space in a gradient beginning at the entry port. Similarly, this pressure differential drives an outward flow of “spent” media from the cell chamber back into the capillary lumena by which wastes are removed. Cells grow in the extracapillary space attachment to the extracapillary walls of the fibers. Typically, oxygen is dissolved into the liquid fraction of the extracapillary space by means of an external reservoir connected to this space via a pump mechanism. Waste products in the intracapillary space may be removed by reverse osmosis in fluid circulated outside of the cell chamber. Such fibers and bioreactors are taught, for example, by U.S. Pat. Nos. 3,821,087; 3,883,393; 3,997,396; 4,087,327; 4,184,922; 4,201,845; 4,220,725; 4,442,206; 4,722,902; 4,804,628; and 4,894,342.

In some embodiments, a solid support attachment moiety is a member of a specific binding pair. Specific binding pairs include, but are not limited to, biotin and streptavidin, digoxin and antidigoxin, and the like. In these embodiments, the support surface is modified to include the complementary member of the specific binding pair. Thus, e.g., where the synthetic attachment polypeptide comprises avidin, the support surface comprises biotin attached thereto.

In some embodiments, a solid support attachment moiety is a reactive group that reacts with a moiety on a solid support surface, and provides for covalent attachment thereto. In some embodiments, a solid support attachment moiety comprises a free sulfhydryl group that reacts with a sulfhydryl group on a support surface, providing for a disulfide linkage of the synthetic attachment polypeptide to the support surface. In some embodiments, a solid support attachment moiety comprises a free amino group that provides for formation of an amide bond with a carboxyl group on the support surface. In other embodiments, a solid support attachment moiety comprises a metal binding moiety that provides for binding to a metal group attached to the support surface. For example, in some embodiments, a solid support attachment moiety comprises a poly-histidine moiety (e.g., (HiS)₆) or a histidine-rich moiety that provides for binding to a metal ion such as Ni⁺², Co⁺², Fe⁺³, Al⁺³, Zn⁺², or Cu⁺².

### Insoluble Supports

The present invention further provides an insoluble support comprising a subject synthetic attachment polypeptide attached to a surface of the insoluble support. The term “insoluble support” is used interchangeably herein with “solid support.” Suitable insoluble supports include, but are not limited to, dextran, polyacrylamide, nylon, polystyrene, calcium alginate, gel structures (e.g., agar gel structures), fibers (e.g., hollow fibers). Insoluble supports are in any of a variety of forms, including, but not limited to, beads (e.g., microcarrier beads); plates (e.g., culture dishes, microtiter plates, and the like); membranes (e.g., synthetic membranes); fibers (e.g., hollow fibers); and the like. Support surfaces include, but are not limited to, dextran, polyacrylamide, nylon, polystyrene, calcium alginate, glass, silica, silicon, collagen, hydroxyapatite, hydrogels, PTFE, polypropylene, nylon, polyacrylamide, and agar gel structures. Support surfaces are in any of a variety of forms, including, but not limited to, beads (e.g., microcarrier beads); plates (e.g., culture dishes, microtiter plates, and the like); membranes (e.g., synthetic membranes); fibers (e.g., hollow fibers); and the like. Suitable support surfaces include particles, e.g., a bead, a microsphere, a nanoparticle, or a colloidal particle. Particle and bead sizes may also be chosen and may have a variety of sizes including wherein the bead is about 5 nanometers to about 500 microns in diameter.

In some embodiments, a subject insoluble support comprises a synthetic attachment polypeptide covalently attached to a surface of the insoluble support. In other embodiments, a subject insoluble support comprises a synthetic attachment polypeptide non-covalently attached to a surface of the insoluble support.

A subject insoluble support generally comprises a plurality of synthetic attachment polypeptides attached to a surface of the insoluble support. In some embodiments, a subject insoluble support comprises a single type of synthetic attachment polypeptide attached to a surface of the insoluble support, e.g., the plurality of synthetic attachment polypeptides comprises a homogeneous population of synthetic attachment polypeptides.

In other embodiments, a subject insoluble support comprises two or more different types of synthetic attachment polypeptides attached to a surface of the insoluble support, e.g., the plurality of synthetic attachment polypeptides comprises a heterogeneous population of synthetic attachment polypeptides. For example, in some embodiments, where the plurality of synthetic attachment polypeptides comprises a heterogeneous population of synthetic attachment polypeptides, the heterogeneous population of synthetic attachment polypeptides comprises two or more different members comprising cell attachment moieties selected from a laminin G domain, a fibronectin domain 2, a fibronectin domain 3, a nidogen G2 domain, a nidogen G3 domain, a vitronectin somatomedin B domain, and a vitronectin carboxyl-terminal domain.

A subject insoluble support comprises a plurality of subject synthetic attachment polypeptides attached to a surface of the insoluble support, where the plurality of attachment polypeptides provides for immobilization of pluripotent cells on the insoluble support at a high density. For example, pluripotent cells are immobilized on a subject insoluble support at a density of from about 10⁴ cells/cm² to about 10⁹ cells/cm², e.g., from about 10⁴ cells/cm² to about 10⁵ cells/cm², from about 10⁵ cells/cm² to about 10⁶ cells/cm², from about 10⁶ cells/cm² to about 10⁷ cells/cm², from about 10⁷ cells/cm² to about 10⁸ cells/cm², or from about 10⁸ cells/cm² to about 10⁹ cells/cm², or greater than 10⁹ Cells/cm².

### Nucleic Acids, Expression Vectors, and Host Cells

A subject synthetic attachment polypeptide is in some embodiments prepared by recombinant methods, using conventional techniques known in the art. The particular sequence and the manner of preparation will be determined by convenience, economics, purity required, and the like.

In some embodiments, a subject synthetic attachment polypeptide is prepared by recombinant methods, e.g., a nucleic acid comprising a nucleotide sequence encoding a subject synthetic attachment polypeptide is introduced into a host cell; and the encoded synthetic attachment polypeptide is synthesized by the host cell. The present invention provides nucleic acids comprising a nucleotide sequence encoding a subject synthetic attachment polypeptide. In many embodiments, the nucleic acid is part of an expression vector. The present invention thus provides expression vectors comprising a subject nucleic acid. The present invention further provides host cells (e.g., in vitro host cells) that comprise a subject nucleic acid or a subject expression vector. Subject nucleic acids, expression vectors, and host cells are described in more detail below.

In many embodiments, an oligonucleotide encoding the amino acid sequence of the synthetic attachment polypeptide is prepared by chemical synthesis, e.g., by using an oligonucleotide synthesizer, wherein oligonucleotides are designed based on the amino acid sequence of the synthetic attachment polypeptide, and in many embodiments, selecting those codons that are favored in the host cell in which the synthetic attachment polypeptide will be produced. For example, several small oligonucleotides coding for portions of the desired polypeptide may be synthesized and assembled by polymerase chain reaction (PCR), ligation or ligation chain reaction (LCR). The individual oligonucleotides typically contain 5′ or 3′ overhangs for complementary assembly. Once assembled, the nucleotide sequence encoding the subject polypeptide is inserted into a recombinant vector and operably linked to control sequences necessary for expression of the desired nucleic acid, and subsequent production of the subject polypeptide, in the desired transformed host cell.

The polypeptide-encoding nucleic acids are generally propagated by placing the nucleic acids in a vector. Viral and non-viral vectors are used, including plasmids. The choice of vector will depend on the type of cell in which propagation is desired and the purpose of propagation. Certain vectors are useful for amplifying and malting large amounts of the desired DNA sequence. Other vectors are particularly useful for production of an encoded polypeptide (“expression vectors”).

A recombinant expression vector is useful for effecting expression of a polypeptide-encoding nucleic acid in a cell, e.g., for production of a known protease-resistant polypeptide variant. The choice of appropriate vector is well within the skill of the art. Many such vectors are available commercially.

Expression vectors are suitable for expression in cells in culture. These vectors will generally include regulatory sequences (“control sequences” or “control regions”) which are necessary to effect the expression of a desired polynucleotide to which they are operably linked.

Expression vectors generally have convenient restriction sites located near the promoter sequence to provide for the insertion of nucleic acid sequences encoding a desired protein or other protein. A selectable marker operative in the expression host may be present. Expression vectors may be used for the production of fusion proteins, where the exogenous fusion peptide provides additional functionality, i.e. increased protein synthesis, stability, reactivity with defined antisera, an enzyme marker, e.g. P-galactosidase, luciferase, etc. Expression cassettes may be prepared that comprise a transcription initiation region, a promoter region (e.g., a promoter that is functional in a eukaryotic cell), a desired polynucleotide, and a transcriptional termination region. After introduction of the DNA, the cells containing the construct may be selected by means of a selectable marker, the cells expanded and then used for expression.

The expression cassettes may be introduced into a variety of vectors, e.g. plasmid, BAC, HAC, YAC, bacteriophage such as lambda, P1, M13, etc., animal or plant viruses, and the like, where the vectors are normally characterized by the ability to provide selection of cells comprising the expression vectors. The vectors may provide for extrachromosomal maintenance, particularly as plasmids or viruses, or for integration into the host chromosome. Where extrachromosomal maintenance is desired, an origin sequence is provided for the replication of the plasmid, which may be low- or high copy-number. A wide variety of markers are available for selection, particularly those which protect against toxins, more particularly against antibiotics. The particular marker that is chosen is selected in accordance with the nature of the host, where in some cases, complementation may be employed with auxotrophic hosts. Introduction of the DNA construct into a host cell may use any convenient method, e.g. conjugation, bacterial transformation, calcium-precipitated DNA, electroporation, fusion, transfection, infection with viral vectors, biolistics, etc.

The present invention further provides genetically modified host cells, which may be isolated host cells (e.g., in vitro host cells), comprising a subject polynucleotide, or, in some embodiments, a subject expression vector. Suitable host cells include prokaryotes such as E. coli, B. subtilis; eukaryotes, including insect cells in combination with baculovirus vectors, yeast cells, such as Saccharomyces cerevisiae, or cells of a higher organism such as vertebrates, including amphibians (e.g., Xenopus laevis oocytes), and mammals, particularly mammals, e.g. COS cells, CHO cells, BEK293 cells, MA-10 cells, and the like, may be used as the expression host cells. Host cells can be used for the purposes of propagating a subject polynucleotide, for production of a subject synthetic attachment polypeptide. In many embodiments, the host cell is a prokaryotic host cell. In particular, the host cell is in many embodiments an E. coli host cell.

The subject synthetic attachment polypeptide can be harvested from the production host cells and then isolated and purified in accordance with conventional methods of recombinant synthesis. A lysate may be prepared of the expression host and the lysate purified using high performance liquid chromatography, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

The present invention provides compositions comprising a subject synthetic attachment polypeptide. Compositions will comprise a subject synthetic attachment polypeptide; and one or more additional components, which are selected based in part on the use of the subject synthetic attachment polypeptide. Suitable additional components include, but are not limited to, salts, buffers, solubilizers, stabilizers, detergents, protease-inhibiting agents, and the like.

### Bioreactor and System

The present invention further provides a bioreactor for culturing pluripotent cells, e.g., ES cells or a multipotent cell. A subject bioreactor comprises a support surface; and a subject synthetic attachment polypeptide bound to the support surface. Any known bioreactor can be modified as described herein, such that a subject synthetic attachment polypeptide is attached to a support surface and provides for immobilization of a pluripotent cell or a multipotent cell. Suitable bioreactors that can be so modified include any of the bioreactors discussed in, e.g., U.S. Pat. Nos. 5,981,211; 5,081,035; 5,126,238; 5,656,421; 4,442,206; 6,670,169; and 6,323,022.

A subject bioreactor comprises a support surface to which a subject synthetic attachment polypeptide is attached. Suitable support surfaces include, but are not limited to, a synthetic membrane, a bead (e.g., a microcarrier bead), a hollow fiber, a gel, a ceramic matrix, and the like.

Suitable bioreactors include the vat type, the packed-column type, and the porous ceramic-matrix type bioreactor. In many embodiments, a bioreactor comprises a cylindrical housing, within which is at least one chamber comprising a support surface to which cells adhere and are immobilized. Typically, a subject bioreactor comprises an inlet and an outlet. An inlet is in fluid communication with a cell chamber and allows introduction of cell culture medium, growth factors, effector molecules, and the like. An outlet provides for release of fluid (e.g., cell culture media) from the cell chamber(s).

In some embodiments, the solid support surface comprises capillary hollow fibers (e.g., configured in elongated bundles of many fibers) having micropores in the fiber walls. The fibers comprise a subject synthetic attachment polypeptide attached thereto. Typically, cells are cultured in a closed chamber into which the fiber bundles are placed. Nutrient aqueous solutions flow freely through the capillary lumen and the hydrostatic pressure of this flow results in an outward radial perfusion of the nutrient liquid into the extracapillary space in a gradient beginning at the entry port. Similarly, this pressure differential drives an outward flow of “spent” media from the cell chamber back into the capillary lumena by which wastes are removed. Cells grow in the extracapillary space attachment to the extracapillary walls of the fibers. Typically, oxygen is dissolved into the liquid fraction of the extracapillary space by means of an external reservoir connected to this space via a pump mechanism. Waste products in the intracapillary space may be removed by reverse osmosis in fluid circulated outside of the cell chamber.

The present invention further provides a system for culturing pluripotent cells, e.g., ES cells. A subject system comprises a subject bioreactor; and one or more additional components such as a fluid control system, a temperature control system. A fluid flow control system may include one or more of a pump; a reservoir; a valve, for controlling the volume, rate, and/or pressure of a fluid, e.g., from a reservoir to the bioreactor. A temperature control system may include one or more of a heating element, a cooling element, a temperature detection means, and the like.

### Method of Culturing Pluripotent or Multipotent Cells

The present invention further provides a method for pluripotent cells, e.g., ES cells or a multipotent cell. The method generally involves culturing pluripotent cells, e.g., ES cells, or a multipotent cell in a subject bioreactor. A pluripotent cell (e.g., an ES cell) or a multipotent cell binds to the cell attachment moiety of a subject synthetic attachment polypeptide, which synthetic attachment polypeptide is attached to a support surface of the bioreactor. By binding to the cell attachment moiety, a pluripotent cell, such as an ES cell, or a multipotent cell becomes immobilized on the support surface.

The density of the immobilized pluripotent cells (e.g., ES cells) or a multipotent cell on a support surface, which support surface has attached thereto a subject synthetic attachment polypeptide, is high. For example, pluripotent cells or a multipotent cells are immobilized on a subject insoluble support at a density of from about 10⁴ cells/cm² to about 10⁹ cells/cm², e.g., from about 10⁴ cells/cm² to about 10⁵ cells/cm², from about 10⁵ cells/cm² to about 10⁶ cells/cm², from about 10⁶ cells/cm² to about 10⁷ cells/cm², from about 10⁷ cells/cm² to about 10⁸ cells/cm², or from about 10⁸ cells/cm² to about 10⁹ cells/cm², or greater than 10⁹ cells/cm².

Cell culture medium is introduced into a subject bioreactor. Suitable cell culture media for maintenance of pluripotent cells and multipotent cells are known in the art. Suitable culture media may contain one or more of the following: isotonic saline, buffer, amino acids, antibiotics, serum or serum replacement, and exogenously added factors. In some embodiments, the culture medium is a serum-free medium. The bioreactor is generally maintained at an appropriate temperature (e.g., 37° C.) and atmosphere (e.g., air plus 5% CO₂).

In some embodiments, cell attachment moieties and/or culture conditions are selected for maintenance of a pluripotent cell (e.g., an ES cell) or a multipotent cell in an undifferentiated state. In other embodiments, cell attachment moieties and/or culture conditions are selected for inducing differentiation of a pluripotent cell (e.g., an ES cell) or a multipotent cell.

Suitable culture media include Dulbecco's Modified Eagle Medium (DMEM), which is commercially available; supplemented with one or more of the following: 3700 mg/l sodium bicarbonate; and 10 ml/l of a 100× antibiotic/antimycotic solution containing 10,000 units penicillin, 10,000 μg streptomycin, and 25 μg amphotericin B/ml.

An example of a suitable medium is DMEM (GIBCO, without sodium pyruvate, with glucose 4500 mg/L) supplemented with 20% fetal bovine serum (FBS) (Hyclone, Utah), β-mercaptoethanol—0.1 mM (GIBCO), non essential amino acids—NEAA 1% (GIBCO), glutamine 2 mM (GIBCO), and penicillin 50 units/ml, streptomycin 50 μg/ml (GIBCO).

The culture medium may be further supplemented with one or more carbohydrates or carbohydrate-containing macromolecules (e.g., glycosaminoglycans, galactosaminoglycans, proteoglycans, and the like). Suitable carbohydrates or carbohydrate-containing macromolecules include chondroitin sulfate, keratan sulfate, hyaluronic acid, dermatan sulfate, heparin sulfate, and the like. Of particular interest in some embodiments, is a chondroitin sulfate. Chondroitin sulfate is made up of linear repeating units containing D-galactosamine and D-glucuronic acid. The molecular weight of chondroitin sulfate ranges from 5,000 to 50,000 daltons and contains about 15 to 150 basic units of D-galactosamine and D-glucuronic acid. Any type of chondroitin sulfate, e.g., chondroitin sulfate A, chondroitin sulfate B, or chondroitin sulfate C, may be used. Chondroitin sulfate A is also referred to as chondroitin-4 sulfate. Chondroitin sulfate B is also known as dermatan sulfate. Chondroitin sulfate C is also referred to as chondroitin-6 sulfate. The concentration range of chondroitin sulfate depends, in part, on the molecular weight of the chondroitin sulfate. As one non-limiting example, at a mean molecular weight of about 4,000 daltons, a suitable concentration range is from about 0.005 mM to about 0.5 mM. In some embodiments, chondroitin sulfate C is added to the culture medium.

The carbohydrates may be added directly to the culture, or added via conjugation to an insoluble surface. When conjugated to a surface, the carbohydrate (e.g. chondroitin sulfate) can be attached directly to the support surface on which the cells are attached, or alternatively provided on a separate insoluble substrate, e.g., a bead or other substrate that may be added to the culture system.

The culture medium may be further supplemented with soluble growth factors which promote stem cell growth or survival or inhibit stem cell differentiation. Examples of such factors include human multipotent stem cell factor, and embryonic stem cell renewal factor. In some embodiments, a cytokine is added to the cell culture medium. Suitable cytokines include, but are not limited to, stem cell factor, FLT3 ligand, interleukin-6, thrombopoietin, interleukin-3, granulocyte colony stimulating factor, granulocyte/macrophage colony stimulating factor, and erythropoietin.

In a specific embodiment, a chemical such as lysophosphatidic acid (LPA) may be used to promote cell attachment and growth. Any usable form of LPA can be used, including non-hydrolyzable LPA, cyclic LPA, non-hydrolyzable cyclic LPA, and/or other LPA derivatives.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A bioreactor, comprising:
a support surface; and
a synthetic attachment polypeptide bound to the support surface wherein the synthetic attachment polypeptide is characterized by a high binding affinity for an embryonic stem cell or a multipotent cell.

2. The bioreactor of claim 1, wherein the synthetic attachment polypeptide comprises a cell attachment domain of from about 10 amino acids to about 500 amino acids in length.

3. The bioreactor of claim 1, wherein the synthetic attachment polypeptide comprises a cell attachment domain of a polypeptide selected from laminin, fibronectin, nidogen, and vitronectin and further wherein lysophosphatidic acid (LPA) is present in the bioreactor.

4. The bioreactor of claim 3, wherein the cell attachment domain is chosen from Laminin G domain, Fibronectin domain 2, Fibronectin domain 3, Nidogen G2 domain, Nidogen G3 domain, Vitronectin somatomedin B domain, and Vitronectin somatomedin carboxyl-terminal domain.

5. A synthetic attachment polypeptide of the formula I:
NH₂—(X₁)ₙ-A-(X₂)ₘ—B—(X₃)ₚ, (I)
wherein A is a moiety that provides for attachment to the surface of an embryonic stem cell;
B is a moiety that provides for attachment to a support surface;
X₁, X₂, and X₃ are each independently any amino acid;
and wherein n, m, and p are each independently 0, or an integer from 1 to about 50.

6. The synthetic attachment polypeptide of claim 5, wherein A is a cell attachment domain of a polypeptide selected from laminin, fibronectin, nidogen, and vitronectin.

7. The synthetic attachment polypeptide of claim 6, wherein the cell attachment domain is chosen from Laminin G domain, Fibronectin domain 2, Fibronectin domain 3, Nidogen G2 domain, Nidogen G3 domain, Vitronectin somatomedin B domain, and Vitronectin somatomedin C terminal domain.

8. An insoluble support comprising the synthetic attachment polypeptide of claim 5 attached to a surface of the insoluble support.

9. The insoluble support of claim 8, wherein the insoluble support is selected from a microcarrier bead, a hollow fiber, a ceramic matrix, and a gel and wherein chondroitin sulfate is attached to a surface of the insoluble support.

10. A nucleic acid comprising a nucleotide sequence encoding the synthetic attachment polypeptide of claim 5.

11. A recombinant vector comprising the nucleic acid of claim 10.

12. The recombinant vector of claim 11, wherein the vector is an expression vector, and wherein the nucleotide sequence encoding the synthetic attachment polypeptide is operably linked to a transcriptional control element.

13. An isolated host cell comprising the recombinant vector of claim 11.

14. The host cell of claim 13, wherein the host cell is a prokaryotic host cell.

15. The host cell of claim 13, wherein the host cell is a eukaryotic host cell.

16. A system for culturing a pluripotent mammalian cell, the system comprising:
the bioreactor of claim 1; and
a fluid control system in fluid communication with the bioreactor.

17. The system of claim 16, further comprising
a temperature control system.

18. A method of culturing a pluripotent cell, the method comprising:
immobilizing a pluripotent cell in the bioreactor of claim 1; and
culturing said pluripotent cell.

19. The method of claim 18, wherein said pluripotent cell is an embryonic stem cell.

20. The method of claim 19, wherein said embryonic stem cell is a human embryonic stem cell.

21. A bioreactor, comprising:
a support surface; and
a synthetic attachment polypeptide having a formula chosen from Formula I and II;
NH₂—(X₁)ₙ-A-(X₂)ₘB—(X₃)ₚ (I)
(X₁)ₙ—B—(X₂)ₘ-A-(X₃)ₚ—NH₂ (II)
wherein A is a moiety that provides for binding affinity to a surface of a pluripotent cell, B is moiety which provides for binding affinity to the support surface, each X is independently an amino acid, n is an integer of from about 1 to about 50, m is an integer of from about 1 to about 50 and p is 0 or an integer from 1 to about 50.

22. The bioreactor of claim 21, wherein the synthetic attachment polypeptide is chosen from Laminin G domain, Fibronectin domain 2, Fibronectin domain 3, Nidogen G2 domain, Nidogen G3 domain, Vitronectin somatomedin B domain, and Vitronectin somatomedin carboxyl-terminal domain.

23. A system for culturing a multipotent mammalian cell, the system comprising:
the bioreactor of claim 1; and
a fluid control system in fluid communication with the bioreactor.

24. The system of claim 23, further comprising a temperature control system.

25. A method of culturing a multipotent cell, the method comprising:
immobilizing a multipotent cell in the bioreactor of claim 1; and
culturing said multipotent cell.

26. The method of claim 25, wherein said multipotent cell is an adult stem cell.

27. The method of claim 26, wherein said adult stem cell is a human adult stem cell.

28. A bioreactor, comprising:
a support surface; and
a synthetic attachment polypeptide having a formula chosen from Formula I and II;
NH₂—(X₁)ₙ-A-(X₂)ₘB—(X₃)ₚ (I)
(X₁)ₙ—B—(X₂)ₘ-A-(X₃)ₚ—NH₂ (II)
wherein A is a moiety that provides for binding affinity to a surface of a multipotent cell, B is moiety which provides for binding affinity to the support surface, each X is independently an amino acid, n is an integer of from about 1 to about 50, m is an integer of from about 1 to about 50 and p is 0 or an integer from 1 to about 50.

29. The bioreactor of claim 28, wherein the synthetic attachment polypeptide is chosen from Laminin G domain, Fibronectin domain 2, Fibronectin domain 3, Nidogen G2 domain, Nidogen G3 domain, Vitronectin somatomedin B domain, and Vitronectin somatomedin carboxyl-terminal domain.


SEQUENCE LISTING (22 sequences)

SEQ ID NO: 1 - protein, 949 aa, Homo sapiens
IRAYQPQISSTNYNTLTLNVKTQEPDNLLFYLGSSTASDFLAVEMRRGRVAFLWDLGSGSTRLEFPDFPIDDNRWHSIHVARFGNIGSLSVKEMSSNQKSPTKTSKSPGTANVLDVNNSTLMFVGGLGGQIKKSPAVKVTHFKGCLGEAFLNGKSIGLWNYIEREGKCRGCFGSSQNEDPSFHFDGSGYSVVEKSLPATVTQIIMLFNTFSPNGLLLYLGSYGTKDFLSIELFRGRVKVMTDLGSGPITLLTDRRYNNGTWYKIAFQRNRKQGVLAVIDAYNTSNKETKQGETPGASSDLNRLDKDPIYVGGLPRSRVVRRGVTTKSFVGCIKNLEISRSTFDLLRNSYGVRKGCLLEPIRSVSFLKGGYIELPPKSLSPESEWLVTFATTNSSGIILAALGGDVEKRGDREEAHVPFFSVMLIGGNIEVHVNPGDGTGLRKALLHAPTGTCSDGQAHSISLVRNRRIITVQLDENNPVEMKLGTLVESRTINVSNLYVGGIPEGEGTSLLTMRRSFHGCIKNLIFNLELLDFNSAVGHEQVDLDTCWLSERPKLAPDAEDSKLLPEPRAFPEQCVVDAALEYVPGAHQFGLTQNSHFILPFNQSAVRKKLSVELSIRTFASSGLIYYMAHQNQADYAVLQLHGGRLHFMFDLGKGRTKVSHPALLSDGKWHTVKTDYVKRKGFITVDGRESPMVTVVGDGTMLDVEGLFYLGGLPSQYQARKIGNITHSIPACIGDVTVNSKQLDKDSPVSAFTVNRCYAVAQEGTYFDGSGYAALVKEGYKVQSDVNITLEFRTSSQNGVLLGISTAKVDAIGLELVDGKVLFHVNNGAGRITAAYEPKTATVLCDGKWHTLQANKSKHRITLIVDGNAVGAESPHTQSTSVDTNNPIYVGGYPAGVKQKCLRSQTSFRGCLRKLALIKSPQVQSFDFSRAFELHGVFLHSCPGTES

SEQ ID NO: 2 - protein, 950 aa, mus musculus
IRAYQPQTSSTNYNTLILNVKTQEPDNLLFYLGSSSSSDFLAVEMRRGKVAFLWDLGSGSTRLEFPEVSINNNRWHSIYITRFGNMGSLSVKEASAAENPPVRTSKSPGPSKVLDINNSTLMFVGGLGGQIKKSPAVKVTHFKGCMGEAFLNGKSIGLWNYIEREGKCNGCFGSSQNEDSSFHFDGSGYAMVEKTLRPTVTQIVILFSTFSPNGLLFYLASNGTKDFLSIELVRGRVKVMVDLGSGPLTLMTDRRYNNGTWYKIAFQRNRKQGLLAVFDAYDTSDKETKQGETPGAASDLNRLEKDLIYVGGLPHSKAVRKGVSSRSYVGCIKNLEISRSTFDLLRNSYGVRKGCALEPIQSVSFLRGGYVEMPPKSLSPESSLLATFATKNSSGILLVALGKDAEEAGGAQAHVPFFSIMLLEGRIEVHVNSGDGTSLRKALLHAPTGSYSDGQEHSISLVRNRRVITIQVDENSPVEMKLGPLTEGKTIDISNLYIGGLPEDKATPMLKMRTSFHGCIKNVVLDAQLLDFTHATGSEQVELDTCLLAEEPMQSLHREHGELPPEPPTLPQPELCAVDTAPGYVAGAHQFGLSQNSHLVLPLNQSDVRKRLQVQLSIRTFASSGLIYYVAHQNQMDYATLQLQEGRLHFMFDLGKGRTKVSHPALLSDGKWHTVKTEYIKRKAFMTVDGQESPSVTVVGNATTLDVERKLYLGGLPSHYRARNIGTITHSIPACIGEIMVNGQQLDKDRPLSASAVDRCYVVAQEGTFFEGSGYAALVKEGYKVRLDLNITLEFRTTSKNGVLLGISSAKVDAIGLEIVDGKVLFHVNNGAGRITATYQPRAARALCDGKWHTLQAHKSKHRIVLTVDGNSVRAESPHTHSTSADTNDPIYVGGYPAHIKQNCLSSRASFRGCVRNLRLSRGSQVQSLDLSRAFDLQGVFPHSCPGPEP

SEQ ID NO: 3 - protein, 2400 aa, Rattus norvegicus
MLRGPGPGRLLLLAVLCLGTSVRCTETGKSKRQAQQIVQPPSPVAVSQSKPGCFDNGKHYQINQQWERTYLGNALVCTCYGGSRGFNCESKPEPEETCFDKYTGNTYKVGDTYERPKDSMIWDCTCIGAGRGRISCTIANRCHEGGQSYKIGDKWRRPHETGGYMLECLCLGNGKGEWTCKPIAEKCFDHAAGTSYVVGETWEKPYQGWMMVDCTCLGEGNGRITCTSRNRCNDQDTRTSYRIGDTWSKKDNRGNLLQCVCTGNGRGEWKCERHVLQSASAGSGSFTDVRTAIYQPQTHPQPAPYGHCVTDSGVVYSVGMQWLKSQGDKQMLCTCLGNGVSCQETAVTQTYGGNSNGEPCVLPFTYNGRTFYSCTTEGRQDGHLWCSTTSNYEQDQKYSFCTDHAVLVQTRGGNSNGALCHFPFLYNNRNYTDCTSEGRRDNMKWCGTTQNYDADQKFGFCPMAAHEEICTTNEGVMYRIGDQWDKQHDLGHMMRCTCVGNGRGEWACIPYSQLRDQCIVDDITYNVNDTFHKRHEEGHMLNCTCFGQGRGRWKCDPIDQCQDSETRTFYQIGDSWEKFVHGVRYQCYCYGRGIGEWHCQPLQTYPGTTGPVQVIITETPSQPNSHPIQWNAPEPSHITKYILRWRPKTSTGRWKEATIPGHLNSYTIKGLTPGVIYEGQLISIQQYGHQEVTRFDFTTSASTPVTSNTVTGETAPFSPVVATSESVTEITASSFVVSWVSASDTVSGFRVEYELSEEGDEPQYLDLPSTATSVNIPDLLPGRKYIVNVYQISEEGKQSLILSTSQTTAPDAPPDPTVDQVDDTSIVVRWSRPQAPITGYRIVYSPSVEGSSTELNLPETANSVTLSDLQPGVQYNITIYAVEENQESTPVFIQQETTGVPRSDDVPAPKDLQFVEVTDVKVTIMWTPPNSAVTGYRVDVLPVNLPGEHGQRLPVNRNTFAEVTGLSPGVTYLFKVFAVHQGRESKPLTAQQTTKLDAPTNLQFVNETDRTVLVTWTPPRARIAGYRLTVGLTRGGQPKQYNVGPMASKYPLRNLQPGSEYTVTLMAVKGNQQSPKATGVFTTLQPLRSIPPYNTEVTETTIVITWTPAPRIGFKLGVRPSQGGEAPREVTSDSGSIVVSGLTPGVEYTYTIQVLRDGQERDAPIVNRVVTPLSPPTNLHLEANPDTGVLTVSWERSTTPDITGYRITTTPTNGQQGTALEEVVHADQSSCTFENLNPGLEYNVSVYTVKDDKESAPISDTVIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIELTNLLVRYSPVKNEEDVAELSISPSDNAVVLTNLLPGTEYLVSVSSVYEQHESIPLRGRQKTGLDSPTGFDSSDVTANSFTVHWVAPRAPITGYIIRHHAEHSAGRPRQDRVPPSRNSITLTNLNPGTEYIVTIIAVNGREESPPLIGQQSTVSDVPRDLEVIASTPTSLLISWEPPAVSVRYYRITYGETGGNSPVQEFTVPGSKSTATINNIKPGADYTITLYAVTGRGDSPASSKPVSINYQTEIDKPSQMQVTDVQDNSISVRWLPSTSPVTGYRVTTAPKNGLGPTKSQTVSPDQTEMTIEGLQPTVEYVVSVYAQNRNGESQPLVQTAVTNIDRPKGLAFTDVDVDSIKIAWESPQGQVSRYRVTYSSPEDGIHELFPAPDGDEDTAELHGLRPGSEYTVSVVALHGGMESQPLIGVQSTAIPAPTNLKFTQVSPTTLTAQWTAPSVKLTGYRVRVTPKEKTGPMKEINLSPDSTSVIVSGLMVATKYEVSVYALKDTLTSRPAQGVVTTLENVSPPRRARVTDATETTITISWRTKTETITGFQVDAIPANGQTPVQRTISPDVRSYTITGLQPGTDYKIHLYTLNDNARSSPVVIDASTAIDAPSNLRFLTTTPNSLLVSWQAPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTNPGYDTENGIQLPGTSHQQPSVGQQMIFEEHGFRRTTPPTAATPVRLRPRPYLPNVDEEVQIGHVPRGDVDYHLYPHVPGLNPNASTGQEALSQTTISWTPFQESSEYIISCQPVGTDEEPLQFQVPGTSTSATLTGLTRGVTYNIIVEALHNQRRHKVREEVVTVGNTVNEGLNQPTDDSCFDPYTVSHYAVGEEWERLSDSGFKLTCQCLGFGSGHFRCDSSKWCHDNGVNYKIGEKWDRQGENGQRMSCTCLGNGKGEFKCDPHEATCYDDGKTYHVGEQWQK

SEQ ID NO: 4 - protein, 2280 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTQGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDPTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNIIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLG

SEQ ID NO: 5 - protein, 94 aa, Artificial Sequence
  synthesized peptide
VSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 6 - protein, 64 aa, Homo sapiens
DITGYRITTTPTNGQQGNSLEEVVHADQSSCTFDNLSPGLEYNVSVYTVKDDKESVPISDTIIP

SEQ ID NO: 7 - protein, 57 aa, mus musculus
AVPPPTDLRFTNIGPDTMRVTWAPPPSIDLTNFLVRYSPVKNEEDVAELSISPSDNA

SEQ ID NO: 8 - protein, 90 aa, Artificial Sequence
  synthesized peptide
EIDKPSQMQVTDVQDNSISVKWLPSSSPVTGYRVTTTPKNGPGPTKTKTAGPDQTEMTIEGLQPTVEYVVSVYAQNPSGESQPLVQTAVT

SEQ ID NO: 9 - protein, 90 aa, Homo sapiens
NIDRPKGLAFTDVDVDSIKIAWESPQGQVSRYRVTYSSPEDGIHELFPAPDGEEDTAELQGLRPGSEYTVSVVALHDDMESQPLIGTQST

SEQ ID NO: 10 - protein, 1247 aa, Homo sapiens
MLASSSRIRAAWTRALLLPLLLAGPVGCLSRQELFPFGPGQGDLELEDGDDFVSPALELSGALRFYDRSDIDAVYVTTNGIIATSEPPAKESHPGLFPPTFGAVAPFLADLDTTDGLGKVYYREDLSPSITQRAAECVHRGFPEISFQPSSAVVVTWESVAPYQGPSRDPDQKGKRNTFQAVLASSDSSSYAIFLYPEDGLQFHTTFSKKENNQVPAVVAFSQGSVGFLWKSNGAYNIFANDRESIENLAKSSNSGQQGVWVFEIGSPATTNGVVPADVILGTEDGAEYDDEDEDYDLATTRLGLEDVGTTPFSYKALRRGGADTYSVPSVLSPRRAATERPLGPPTERTRSFQLAVETFHQQHPQVIDVDEVEETGVVFSYNTDSRQTCANNRHQCSVHAECRDYATGFCCSCVAGYTGNGRQCVAEGSPQRVNGKVKGRIFVGSSQVPIVFENTDLHSYVVMNHGRSYTAISTIPETVGYSLLPLAPVGGIIGWMFAVEQDGFKNGFSITGGEFTRQAEVTFVGHPGNLVIKQRFSGIDEHGHLTIDTELEGRVPQIPFGSSVHIEPYTELYHYSTSVITSSSTREYTVTEPERDGASPSRIYTYQWRQTITFQECVHDDSRPALPSTQQLSVDSVFVLYNQEEKILRYAFSNSIGPVREGSPDALQNPCYIGTHGCDTNAACRPGPRTQFTCECSIGFRGDGRTCYDIDECSEQPSVCGSHTICNNHPGTFRCECVEGYQFSDEGTCVAVVDQRPINYCETGLHNCDIPQRAQCIYTGGSSYTCSCLPGFSGDGQACQDVDECQPSRCHPDAFCYNTPGSFTCQCKPGYQGDGFRCVPGEVEKTRCQHEREHILGAAGATDPQRPIPPGLFVPECDAHGHYAPTQCHGSTGYCWCVDRDGREVEGTRTRPGMTPPCLSTVAPPIHQGPAVPTAVIPLPPGTHLLFAQTGKIERLPLEGNTMRKTEAKAFLHVPAKVIIGLAFDCVDKMVYWTDITEPSIGRASLHGGEPTTIIRQDLGSPEGIAVDHLGRNIFWTDSNLDRIEVAKLDGTQRRVLFETDLVNPRGIVTDSVRGNLYWTDWNRDNPKIETSYMDGTNRRILVQDDLGLPNGLTFDAFSSQLCWVDAGTNRAECLNPSQPSRRKALEGLQYPFAVTSYGKNLYFTDWKMNSVVALDLAISKETDAFQPHKQTRLYGITTALSQCPQGHNYCSVNNGGCTHLCLATPGSRTCRCPDNTLGVDCIERK

SEQ ID NO: 11 - protein, 1245 aa, mus musculus
MLDASGCSWAMWTWALLQLLLLVGPGGCLNRQELFPFGPGQGDLELEAGDDVVSPSLELIGELSFYDRTDITSVYVTTNGIIAMSEPPATEYHPGTFPPSFGSVAPFLADLDTTDGLGNVYYREDLSPFIIQMAAEYVQRGFPEVSFQPTSVVVVTWESVAPYGGPSSSPAEEGKRNTFQAVLASSNSSSYAIFLYPEDGLQFFTTFSKKDESQVPAVVGFSKGLVGFLWKSNGAYNIFANDRESIENLAKSSNAGHQGVWVFEIGSPATAKGVVSADVNLDLDDDGADYEDEDYDLVTSHLGLEDVATPSPSHSPRRGYPDPHNVPRILSPGYEATERPRGVPTERTRSFQLPAERFPQHHPQVIDVDEVEETGVVFSYNTGSQQTCANNRHQCSVHAECRDYATGFCCRCVANYTGNGRQCVAEGSPQRVNGKVKGRIFVGSSQVPVVFENTDLHSYVVMNHGRSYTAISTIPETVGYSLLPLAPIGGIIGWMFAVEQDGFKNGFSITGGEFTRQAEVTFLGHPGKLVLKQQFSGIDEHGHLTISTELEGRVPQIPYGASVHIEPYTELYHYSSSVITSSSTREYTVMEPDQDGAAPSHTHIYQWRQTITFQECAHDDARPALPSTQQLSVDSVFVLYNKEERILRYALSNSIGPVRDGSPDALQNPCYIGTHGCDSNAACRPGPGTQFTCECSIGFRGDGQTCYDIDECSEQPSRCGNHAVCNNLPGTFRCECVEGYHFSDRGTCVAAEDQRPINYCETGLHNCDIPQRAQCIYMGGSSYTCSCLPGFSGDGRACRDVDECQHSRCHPDAFCYNTPGSFTCQCKPGYQGDGFRCMPGEVSKTRCQLEREHILGAAGGADAQRPTLQGMFVPQCDEYGHYVPTQCHHSTGYCWCVDRDGRELEGSRTPPGMRPPCLSTVAPPIHQGPVVPTAVIPLPPGTHLLFAQTGKIERLPLERNTMKKTERKAFLHIPAKVIIGLAFDCVDKVVYWTDISEPSIGRASLHGGEPTTIIRQDLGSPEGIALDHLGRTIFWTDSQLDRIEVAKMDGTQRRVLFDTGLVNPRGIVTDPVRGNLYWTDWNRDNPKIETSHMDGTNRRILAQDNLGLPNGLTFDAFSSQLCWVDAGTHRAECLNPAQPGRRKVLEGLQYPFAVTSYGKNLYYTDWKTNSVIAMDLAISKEMDTFHPHKQTRLYGITIALSQCPQGHNYCSVNNGGCTHLCLPTPGSRTCRCPDNTLGVDCIERK

SEQ ID NO: 12 - protein, 283 aa, Artificial Sequence
  synthesized peptide
RQTCANNRHQCSVHAECRDYATGFCCSCVAGYTGNGRQCVAEGSPQRVNGKVKGRIFVGSSQVPIVFENTDLHSYVVMNHGRSYTAISTIPETVGYSLLPLAPVGGIIGWMFAVEQDGFKNGFSITGGEFTRQAEVTFVGHPGNLVIKQRFSGIDEHGHLTIDTELEGRVPQIPFGSSVHIEPYTELYHYSTSVITSSSTREYTVTEPERDGASPSRIYTYQWRQTITFQECVHDDSRPALPSTQQLSVDSVFVLYNQEEKILRYALSNSIGPVREGSPDALQ

SEQ ID NO: 13 - protein, 39 aa, Artificial Sequence
  synthesized peptide
QTCANNRHQCSVHAECRDYATGFCCRCVANYTGNGRQCV

SEQ ID NO: 14 - protein, 401 aa, Artificial Sequence
  synthesized peptide
TRCQHEREHILGAAGATDPQRPIPPGLFVPECDAHGHYAPTQCHGSTGYCWCVDRDGREVEGTRTRPGMTPPCLSTVAPPIHQGPAVPTAVIPLPPGTHLLFAQTGKIERLPLEGNTMRKTEAKAFLHVPAKVIIGLAFDCVDKMVYWTDITEPSIGRASLHGGEPTTIIRQDLGSPEGIAVDHLGRNIFWTDSNLDRIEVAKLDGTQRRVLFETDLVNPRGIVTDSVRGNLYWTDWNRDNPKIETSYMDGTNRRILVQDDLGLPNGLHFDAFSSQLCWVDAGTNRAECLNPSQPSRRKALEGLQYPFAVTSYGKNLYFTDWKMNSVVALDLAISKETDAFQPHKQTRLYGITTALSQCPQGHNYCSVNNGGCTHLCLATPGSRTCRCPDNTLGVDCIERK

SEQ ID NO: 15 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 16 - protein, 51 aa, Homo sapiens
DQESCKGRCTEGFNVDKKCQCDELCSYYQSCCTDYTAECKPQVTRGDVFTM

SEQ ID NO: 17 - protein, 6 aa, Artificial Sequence
  synthesized peptide
AAAGGM

SEQ ID NO: 18 - protein, 14 aa, Artificial Sequence
  synthesized peptide
AAAGGMPPAAAGGM

SEQ ID NO: 19 - protein, 6 aa, Artificial Sequence
  synthesized peptide
AAAGGM

SEQ ID NO: 20 - protein, 8 aa, Artificial Sequence
  synthesized peptide
PPAAAGGM

SEQ ID NO: 21 - protein, 4 aa, Artificial Sequence
  synthesized peptide
IEGR

SEQ ID NO: 22 - protein, 6 aa, Artificial Sequence
  synthesized peptide
GGKGGK